(19) United States
(12) Patent Application Publication
(10) Pub. No.: US 2004/0097803 A1
(43) Pub. Date: May 20, 2004

(54) 3-D catheter localization using permanent magnets with asymmetrical properties about their longitudinal axis

(76) Inventor: Dorin Panescu, San Jose, CA (US)

Correspondence Address: Michael J. Bolan, Bingham McCutchen LLP, 18th Floor, Three Embarcadero Center, San Francisco, CA, 94111, US

(21) Appl. No.: 10/300,706

(22) Filed: Nov. 20, 2002

(51) Int. Cl. A61B 5/05

(52) U.S. Cl. 600/424

(57) ABSTRACT

The present invention provides systems and methods for localizing a catheter. One or more permanent magnets are mounted to a catheter. The permanent magnet(s) generate a magnetic field that is asymmetrical about the longitudinal axis of the catheter. One or more magnetic sensors are used to sense the magnetic field. Processing circuitry is configured for determining the roll of the catheter based on the sensed magnetic field. The processing circuitry can also determine the positional coordinates and/or the pitch and yaw of the catheter based on this sensed magnetic field.

### FIELD OF THE INVENTION

The present inventions generally relate to the navigation of medical imaging devices, and more particularly to the magnetic localization of catheters.

### BACKGROUND OF THE INVENTION

Physicians make use of catheters today in medical procedures to gain access into interior regions of the body for diagnostic and therapeutic purposes. It is important for the physician to be able to precisely position the catheter within the body to gain contact with a desired tissue location. The need for precise control over the catheter is especially critical during procedures that ablate myocardial tissue from within the heart. These procedures, called ablation therapy, are used to treat cardiac rhythm disturbances.

During these procedures, a physician introduces a mapping catheter into the heart through a venous or arterial access. Using the electrode(s) of the mapping catheter, the physician examines the propagation of electrical impulses in heart tissue to locate aberrant conductive pathways on the endocardium and to identify the arrhythmia foci. Once the endocardial mapping identifies the foci, an ablation catheter is introduced into the heart. The physician steers the ablation electrode of the catheter into contact with the foci, and directs energy from the electrode through the myocardial tissue either to an indifferent electrode (in a uni-polar electrode arrangement) or an adjacent electrode (in a bipolar electrode arrangement) to ablate the tissue.

Currently, the locations of the ablation and mapping catheters within the patient's body are routinely detected by the use of x-ray fluoroscopic imaging equipment. In this technique, a fluoroscopic image of the catheter (or at least radiopaque bands located on the catheter) and surrounding anatomical landmarks (with or without the use of contrast media) in the body are taken and displayed to the physician. The fluoroscopic image enables the physician to ascertain the position of the catheter within the body and maneuver the catheter within the heart. Excessive exposure to x-ray dosages by both the patient and physician, however, can be harmful. As a result, alternative catheter tracking techniques have been developed.

In one tracking technique, ultrasound pulses are transmitted between ultrasound transducers mounted on reference catheters, as well as the distal end of the catheter to be tracked. The relative distances between the transducers are calculated using the “time of flight” and velocity of the ultrasound pulses. The distances between the reference transducers are triangulated to establish a three-dimensional coordinate system, and then the distances between the tracking transducers and the reference transducers are triangulated to determine the coordinates of the reference transducers, and thus the distal end of the catheter, within the coordinate system. Additional details on this type of tracking technique can be found in U.S. patent application Ser. No. 09/128,304, entitled “A dynamically alterable three-dimensional graphical model of a body region,” which is hereby fully and expressly incorporated herein by reference.

In another tracking technique, three orthogonal magnetic fields are transmitted from external antennas to an array of magnetic field sensors mounted on the distal end of catheter to be tracked. Each magnetic field sensors measures the strength of a respective one of the orthogonal magnetic fields. The magnetic field strength measurements are used to compute distance vectors between the magnetic location array and the centers of the antennas, which are then deconstructed into their x, y, and z components in order to compute the position and orientation of each magnetic location array in the 3-D coordinate system. Additional details on this type of tracking technique can be found in U.S. Pat. No. 5,391,199 to Ben-Haim, entitled “Apparatus and Method for Treating Cardiac Arrhythmias,” which is hereby fully and expressly incorporated herein by reference.

In still another tracking technique, three electrode pairs are placed on the patient's skin in mutually orthogonal directions to establish a three-dimensional coordinate system. A reference potential electrode is placed on the patient's skin in order to establish a reference potential. The three electrode pairs are driven to transmit three orthogonal multiplexed alternating currents across the patient's body. A location electrode mounted on the distal end of the catheter to be tracked measures a voltage (i.e. potential) in the body associated with each of the three orthogonal currents. The voltage value associated with each current indicates the relative distance between the location electrode and the corresponding electrode pair. The x, y, and z coordinates of the location electrode, and thus the distal end of the catheter, are then computed in the three-dimensional coordinate system using the referenced voltage measurements. Additional details on this type of tracking technique can be found in U.S. Pat. No. 5,983,126, entitled “Catheter Location System and Method”, which is fully and expressly incorporated herein by reference.

The above-described ultrasound-, magnetic-, and voltage-based tracking techniques are generally sufficient for tracking catheters, such as cardiac mapping and ablation catheters. All of these techniques, however, require signal wires to be routed from the sensors at the distal end of the catheter to the handle of the catheter. This requirement takes up valuable space within the catheter and adds complexity to the handle.

As such, there remains a need to reduce or completely eliminate the wires that would otherwise have to be routed from the distal end of a catheter to provide tracking capability to the catheter.

### SUMMARY OF THE INVENTION

In accordance with a first aspect of the present inventions, a catheter tracking system comprises a catheter having a longitudinal axis, and one or more permanent magnets mounted to the catheter. The catheter may comprise an operative element (such as, e.g., an ablation electrode, a cardiac mapping electrode, imaging element, or drug delivery stiletto). The permanent magnet(s) exhibit a asymmetrical magnetic field about the longitudinal axis. This can be accomplished using a variety of manners. For example, in one preferred embodiment, the permanent magnets comprises a first magnet (e.g., a cylindrical magnet) with opposite poles aligned parallel with the longitudinal axis of the catheter, and a second magnet (e.g., a disk-shaped magnet) with opposite poles aligned perpendicular to the longitudinal axis of the catheter. As another example, the permanent magnet(s) can comprise a torroidal magnet with opposite poles aligned with perpendicular to the longitudinal axis of the catheter, wherein non-magnetic material is disposed between the opposite poles. As can be appreciated, no wires are required to actuate the permanent magnet(s).

The permanent magnet(s) can be composed of any suitable magnetic material, but in the preferred embodiment, are composed of a rare-earth or plastic magnetic material. In order to reduce the effects of the Earth's magnetic field, the magnetic field intensity exhibited by the permanent magnet(s) are preferably much greater than that of the Earth's magnetic field, e.g., greater than 20 or, even 40, times the magnetic field intensity of the Earth.

The catheter tracking system further comprises one or more magnetic sensors (e.g., coil, Hall-effect or Giant Magnetoresistive (GMR) sensors) configured for sensing the magnetic field. In the preferred embodiment, the magnetic sensor(s) are mounted external to the patient, e.g., by affixing them to a patient table or fixtures associated with the table, or even on the patient. The magnetic sensor(s) can, however, be mounted internal to the patient as well, e.g., by placing them on one or more reference catheters.

The localization system further comprises processing circuitry configured for determining the roll of the catheter body about the longitudinal axis based on the sensed magnetic field. The processing circuitry can be further configured to determine the positional coordinates and/or pitch and yaw of the catheter within a three-dimensional coordinate system based on the sensed magnetic field. By way of non-limiting example, the processing circuitry can include a table with magnetic field intensities as inputs, and the roll of the catheter as an output.

In accordance with a second aspect of the present inventions, a method of tracking a catheter is provided. The method comprises introducing the catheter into the body of a patient. The method may further comprise performing a medical procedure with the catheter, e.g., ablating tissue, mapping cardiac tissue, imaging tissue, delivering therapeutic agents, etc. The method further comprises emitting a magnetic field from a permanent magnet mounted to the catheter, wherein the magnetic field is asymmetrical about a longitudinal axis of the catheter. The method further comprises sensing the magnetic field. In the preferred method, the magnetic field can be sensed external to the body of the patient, but can be sensed internal to the body of the patient as well. The method further comprises determining the roll of the catheter about the longitudinal axis based on the sensed magnetic field. Optionally, the positional coordinates and/or the pitch and yaw of the catheter can be determined based on the sensed magnetic field. The method may optionally comprise storing a table having magnetic field intensities as inputs, and the roll of the catheter as output, in which case, the determined roll of the catheter can be based on this table.

Other objects and features of the present invention will become apparent from consideration of the following description taken in conjunction with the accompanying drawings.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a functional block diagram of one preferred embodiment of a catheter localization system constructed in accordance with the present inventions, particularly showing the mounting of magnetic sensors in association with a patient table;

FIG. 2 is a cross-sectional view of an ablation catheter used in the catheter localization system of FIG. 1;

FIG. 3 is a lateral view of a permanent magnet array mounted to the catheter of FIG. 2;

FIG. 4 is a front view of the permanent magnet array of FIG. 3;

FIG. 5 is a top view of the permanent magnet array of FIG. 3;

FIG. 6 is a plan view of an alternative method of mounting the magnetic sensors of FIG. 1 on reference catheters;

FIG. 7 is a cross-sectional view of a therapeutic agent delivery catheter that can be used in the catheter localization system of FIG. 1;

FIG. 8 is a lateral view of a permanent magnet mounted to the catheter of FIG. 7;

FIG. 9 is a front view of the permanent magnet of FIG. 8; and

FIG. 10 is a top view of the permanent magnet of FIG. 8.

### DETAILED DESCRIPTION OF THE PREFERRED EMBODIMENTS

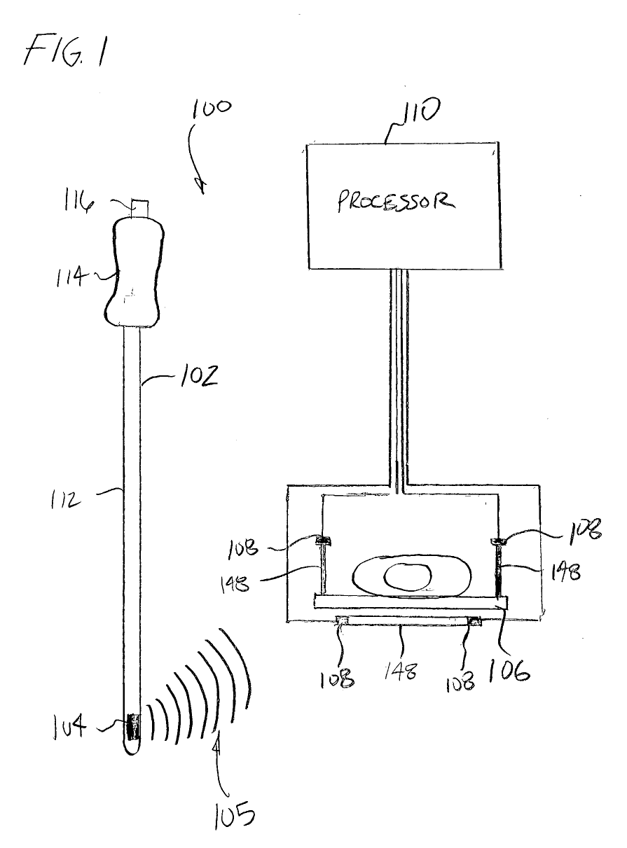

Referring to FIG. 1, an exemplary catheter localization system 100 constructed in accordance with the present inventions is shown. The localization system 100 generally comprises a catheter 102 with a distally mounted permanent magnet array 104 that emits a magnetic field 105, a patient table 106 for supporting a patient, a plurality of magnetic sensors 108 associated with the patient table 106 for sensing the magnetic field 105 emitted by the magnet array 104, and a processor 110 electrically coupled to the plurality of magnetic sensors 108 and configured for determining the position and orientation of the magnet array 104, and thus the distal end of the catheter 102, based on the sensed magnetic field 105.

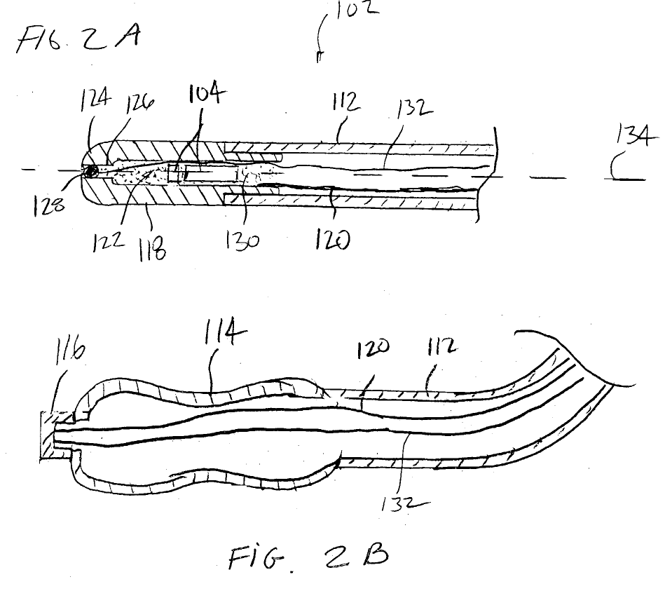

Referring further to FIG. 2, the catheter 102 comprises an elongate catheter body 112 composed of a suitable biocompatible flexible material, such as polyurethane or polyethylene. In the illustrated embodiment, the catheter 102 provides ablation therapy. In particular, the catheter 102 comprises a proximally mounted handle assembly 114 that includes a radio frequency connector 116 that can be coupled to a radio frequency (RF) generator (not shown), and a distally mounted ablation tip electrode 118 that is composed of a suitable biocompatible electrically conductive material, such as 90/10 platinum iridium alloy. The catheter 102 further comprises an RF wire 120 suitably connected to the ablation electrode 118, e.g., by soldering. The RF wire 120 extends proximally the length of the catheter body 112 to the connector 116.

The ablation electrode 118 comprises a cavity 122 in which the permanent magnet array 104 is mounted, and a distal tip 124 that includes an open channel 126 in which a thermistor 128 is mounted. The permanent magnet array 104 and thermistor 128 is suitably affixed within the cavity 122 and channel 126 using a potting material 130, such as an epoxy or ultraviolet (UV) adhesive. The thermistor 128 comprises signal wires 132 that, like the RF wire 120, extend proximally the length of the catheter body 112 to the connector 116.

The permanent magnet array 104 is composed of a magnetic material that generates a relatively high magnetic field strength per unit volume, so that the magnet array 104 can be small enough to be located within the catheter 102, while generating a magnetic field that can be sensed from outside the patient's body without severe degradation by the Earth's magnetic field. For example, the magnetic field strength of the magnet array 104 may be 20 times, and preferably 40 times, the strength of the Earth's magnetic field. Suitable magnetic materials include rare-earth magnetic material (such as, e.g., samarium cobalt and neodymium iron boron) or plastic magnetic materials. Thus, it can be appreciated that no wires are required to be routed from the permanent magnet array 104 back to the handle assembly 114, allowing reduction in the size of the catheter body 112 and simplifying the design of the handle assembly 114.

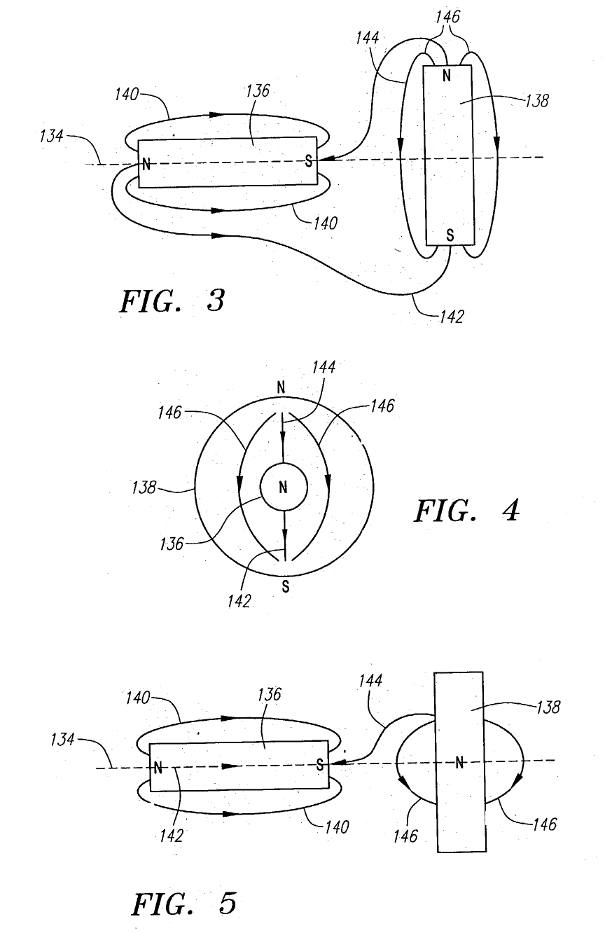

Referring now to FIGS. 3-5, the permanent magnet array 104 comprises two magnets: a cylindrical dipole magnet 136 having North and South poles that align with the longitudinal axis 134 of the catheter body 112, and a disk-shaped dipole magnet 138 having North and South poles that align perpendicularly with the longitudinal axis 134 of the catheter body 112. By itself, the cylindrical dipole magnet 134 will generate a magnetic field that is generally symmetrical about the longitudinal axis 134 of the catheter body 112. The presence of the disk-shaped dipole magnet 138 disturbs the symmetrical magnetic field of the cylindrical dipole magnet 136, resulting in a magnetic field that is asymmetrical about the longitudinal axis 134 of the catheter 102, as illustrated by the exemplary fields lines 140-146 shown in FIGS. 3-5. Magnetic field lines 140 extend from the North pole of the cylindrical dipole magnet 136 to the South pole of the cylindrical dipole magnet 136. Magnetic field lines 142 extend from the North pole of the cylindrical dipole magnet 136 to the South pole of the disk-shaped dipole magnet 138. Magnetic field lines 144 extend from the North pole of the disk-shaped dipole magnet 138 to the South pole of the cylindrical dipole magnet 136. Magnetic field lines 146 extend from the North pole of the disk-shaped dipole magnet 138 to the South pole of the disk-shaped dipole magnet 138. This asymmetry in the magnetic field 105 allows the roll of the catheter 102 to be more easily determined.

Referring back to FIG. 1, the magnetic sensors 108 are affixed to the patient table 106, or to fixtures 148 associated with the patient table 106, in a known three-dimensional arrangement, so that a three-dimensional coordinate system can be established. Although only six magnetic sensors 108 are theoretically required to obtain three positional coordinates (x, y, z) and three angular positions (pitch, yaw, and roll) of the magnet array 104, nine to twelve magnetic sensors 108 are preferably provided to improve the accuracy of the calculations. The magnetic sensors 108 can take any form, e.g., coil, Hall-effect, flux-gate, core-inductive, squid, magneto-resistive, nuclear precession sensors and the like. In the illustrated embodiment, giant magneto-resistive (GMR) sensors are used for their high sensitivity and reduced size.

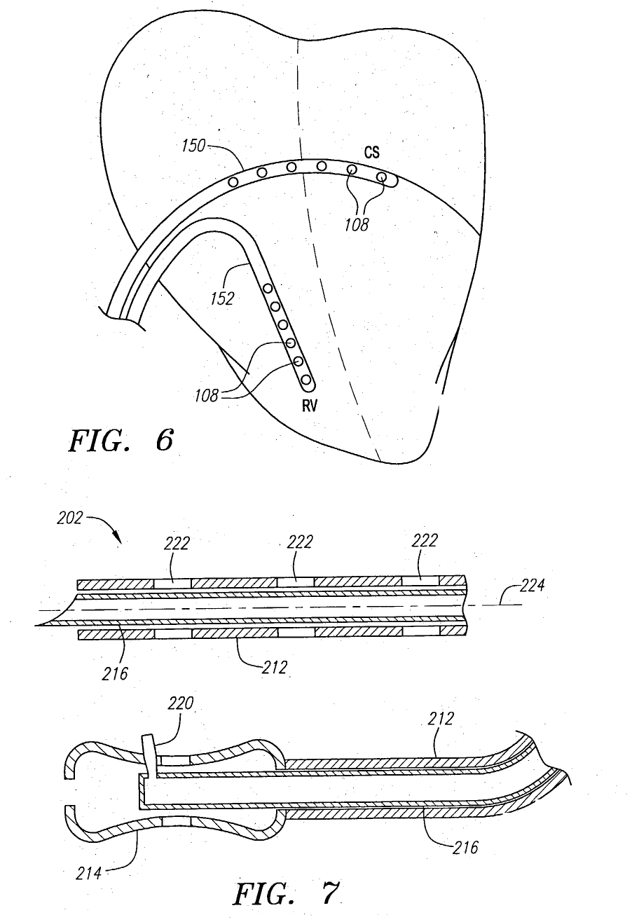

It should be noted that the magnet sensors 108 need not necessarily be associated with the patient table 106. For example, the magnetic sensors 108 can be mounted externally on the patient. As another example, the magnetic sensors 108 can be mounted to one or more catheters that can be placed anywhere within the body (preferably, a known location) that arranges the magnetic sensors 108 in three-dimensional space, and that allows the magnetic sensors 108 to sense the magnetic field 105 from the magnet array 104. For example, if the body tissue to be treated is heart tissue, as illustrated in FIG. 6, the first two dimensions of the coordinate system are provided by placing a reference catheter 150 within the coronary sinus (CS) to arrange its magnetic sensors 108 in a two-dimensional plane, and the third dimension is provided by placing another reference catheter 152 within the right ventricular (RV) apex to arrange its magnetic, sensors 108 off of the two-dimensional plane.

If the magnetic sensors 108 are mounted to the patient or on reference catheters in initially unknown relative positions, the positions of the magnetic sensors 108 can be determined using an independent localization system. For example, ultrasound pulses can be transmitted between ultrasound transducers mounted adjacent the magnetic sensors 108 on the patient's body or on the reference catheters. The distances between the ultrasound transducers can be determined based on the “time of flight” of the pulses, and then triangulated to determine their relative positions, and thus, the relative positions of the magnetic sensors 108. Further details on ultrasound-based localization systems are disclosed in U.S. patent application Ser. No. 09/128,304, which has previously been incorporated by reference.

As previously described, the processor 110 is configured for determining the positional coordinates and angular orientation of the magnet array 104, and thus, the distal end of the catheter body 112, in a three-dimensional coordinate system. Specifically, a table containing nine to twelve inputs defined by the magnetic field intensities sensed by the magnetic sensors 108, and six outputs providing the three positional coordinates (x, y, z) and three angular orientations (pitch, yaw, roll) of the magnet array 104, is stored in the processor 110. This table can be generating using a variety of suitable means. For example, the actual magnetic field intensities sensed by fixed test magnetic sensors can be measured while moving a test permanent magnet array in various positional coordinates and angular orientations. Alternatively, given various permutations of the positional coordinates and angular orientations of a theoretical magnet, the magnetic field intensities at the fixed magnetic sensor positions can be calculated using the Biot-Savart law or numerically modeled. Thus, during actual operation, the processor 110 can recall the table and quickly determine the positional coordinates and angular orientations of the magnet array 104 based on the magnetic field intensities sensed by the magnetic sensors 108.

Because the magnetic field 105 generated by any one magnet array 104 may vary slightly, each magnet array 104 is preferably calibrated to generate a calibration factor prior to installation within its respective catheter body 112. The processor 110 can then use the calibration factor shipped with the catheter 102 to adjust the values contained in the table. For example, the calibration factor can be stamped or otherwise placed on the catheter 102 or documentation associated with the catheter. As another example, the calibration factor can be electronically stored within the catheter 102. Details on electronically storing and retrieving unique information within a catheter are disclosed in U.S. patent application Ser. No. 08/738,814, filed Oct. 28, 1996, entitled “Systems and Methods for Identifying Physical, Mechanical and Functional Attributes of Multiple Electrode Arrays,” which is hereby fully and expressly incorporated herein by reference.

It should be noted that the Earth's magnetic field can introduce errors in determining the exact position and orientation of the magnet array 104. The adverse effects of the Earth's magnetic field, however, can be accounted for using any of a variety of compensation techniques. For example, one or more magnetic sensors located far away from the patient table 106 can be dedicated to the sole purpose of measuring the Earth's magnetic field in real time. This measured information can then be used to determine the Earth's magnetic field vector, which can subtracted from, or used to offset, the measurements sensed by the remaining magnetic sensors 108. As another example, prior to introducing the catheter 102 within the patient, the sensor measurements from all of the magnetic sensors 108 can be adjusted so that they read “zero,” thereby removing any effects of the Earth's magnetic field on the sensor measurements when the catheter 102 is introduced into the patient. A combination of these two processes can also be used to compensate for the Earth's magnetic field.

Although only an ablation catheter has been described so far, other types of catheters and permanent magnets can be used in the localization system 100. For example, FIG. 7 illustrates a delivery catheter 202 for delivering therapeutic agents, such as drugs or deoxyribonucleic acid (DNA). Like the previously described ablation catheter 102, the delivery catheter 202 comprises an elongate catheter body 212 composed of a suitable biocompatible flexible material, such as polyurethane or polyethylene. In this case, however, the diameter of the catheter body 212 is smaller than that of the ablation catheter 102. The delivery catheter 202 further comprises a central lumen 214, a drug delivery stiletto 216 slidably disposed within the central lumen 214, and a proximally mounted handle assembly 218 that includes a drug port 220 in fluid communication with the stiletto 216. As can be appreciated, manipulation of the handle assembly 114 can deploy the stiletto 216 out from the distal end of the catheter body 212, and delivery of a therapeutic agent from the stiletto can be achieved by introducing the therapeutic agent through the drug port 220 on the handle assembly 214.

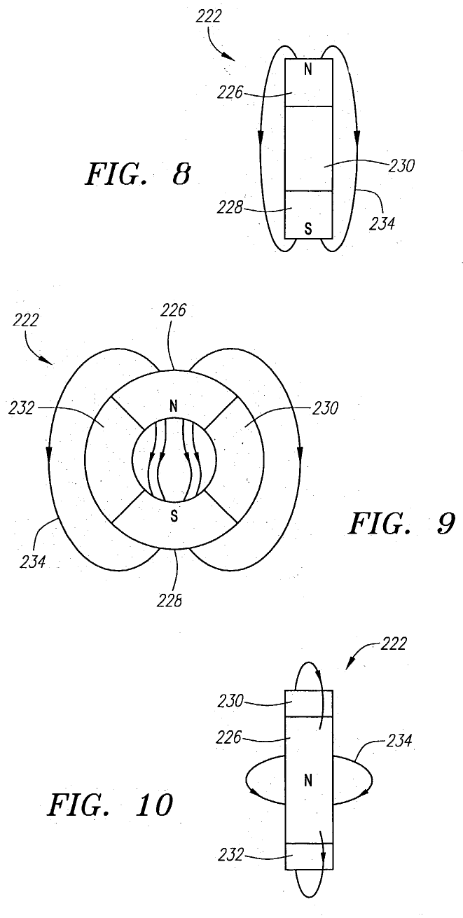

The catheter 202 further comprises a plurality of permanent magnets 222 distributed along the distal end of the catheter body 212. Notably, the torroidal shape of the permanent magnets 222 allows them to be mounted on the outside of the catheter body 212, thereby allowing localization of the very small diameter catheter 202. Turning now to FIGS. 8-10, each permanent magnet 222, like the magnets of the previously described permanent magnet array 104, is composed of a magnetic material that generates a relatively high magnetic field strength per unit volume. The magnet 222 is a dipole magnet having North and South poles that align perpendicularly with the longitudinal axis 224 of the catheter body 212. If the magnet 222 is composed entirely of the magnetic material, it will generate a magnetic field that is generally symmetrical about the longitudinal axis 224 of the catheter body 212. The magnet 222, however, comprises magnetic top and bottom segments 226 and 228 and non-magnetic central segments 230 and 232, resulting in a magnetic field that is asymmetrical about the longitudinal axis 224 of the catheter 202, as illustrated by the exemplary field lines 234 shown in FIGS. 8-10.

The processor 110 will function in the same manner previously described, with the exception that the values stored in the table will be unique to each of the torroidal-shaped magnets 222. The processor 110 can additionally determine the curvature of the catheter body 212 by extrapolating the determined positions of the magnets 222 based on the known structure of the catheter body and the positional relationship between the magnets 222.

It should be noted that other types of catheters besides ablation and drug delivery catheters (e.g., cardiac mapping catheters and imaging catheters) can include permanent magnets for localization purposes. In the case of imaging catheters, three-dimensional and four dimensional (fourth dimension is time) images can be constructed based on the determined position and orientation of the permanent magnet. Further details on the construction of three- and four-dimensional images using an imaging catheter is described in U.S. patent application Ser. No. 10/012,293, entitled “Systems and Methods for Guiding Catheters Using Registered Images,” which is hereby fully and expressly incorporated by reference.

In the foregoing specification, the invention has been described with reference to a specific embodiment thereof. It will, however, be evident that various modifications and changes may be made thereto without departing from the broader spirit and scope of the invention. For example, the reader is to understand that the specific ordering and combination of process actions shown in the process flow diagrams described herein is merely illustrative, and the invention can be performed using different or additional process actions, or a different combination or ordering of process actions. As another example, features known to those of skill in the art can be added to the embodiment. Other processing steps known to those of ordinary skill in the art may similarly be incorporated as desired. Additionally and obviously, features may be added or subtracted as desired. Accordingly, the invention is not to be restricted except in light of the attached claims and their equivalents.

What is claimed is:

1. A catheter tracking system, comprising:
a catheter having a longitudinal axis;
one or more permanent magnets mounted to the catheter, the one or more permanent magnets exhibiting a asymmetrical magnetic field about the longitudinal axis;
one or more magnetic sensors configured for sensing the magnetic field; and
processing circuitry configured for determining the roll of the catheter about the longitudinal axis based on the sensed magnetic field.

2. The system of claim 1, wherein the catheter comprises an operative element.

3. The system of claim 2, wherein the operative element comprises an ablation electrode.

4. The system of claim 2, wherein the operative element comprises a cardiac mapping electrode.

5. The system of claim 2, wherein the operative element comprises an imaging element.

6. The system of claim 2, wherein the operative element comprises a therapeutic agent delivery stiletto.

7. The system of claim 1, wherein the one or more permanent magnets is composed of rare-earth magnetic material.

8. The system of claim 1, wherein the one or more permanent magnets is composed of plastic magnetic material.

9. The system of claim 1, wherein the one or more permanent magnets exhibits a magnetic field intensity that is at least 20 times greater than the magnetic field intensity of Earth.

10. The system of claim 1, wherein the one or more permanent magnets exhibits a magnetic field intensity that is at least 40 times greater than the magnetic field intensity of Earth.

11. The system of claim 1, wherein the one or more permanent magnets comprises a plurality of magnets.

12. The system of claim 1, wherein the one or more permanent magnets comprises a single magnet.

13. The system of claim 1, wherein the one or more permanent magnets comprises a first magnet with opposite poles aligned parallel with the longitudinal axis of the catheter, and a second magnet with opposite poles aligned perpendicular to the longitudinal axis of the catheter.

14. The system of claim 13, wherein the first magnet is a cylindrical magnet and the second magnet is a disk-shaped magnet.

15. The system of claim 1, wherein the one or more permanent magnets comprises a torroidal magnet with opposite poles aligned perpendicular to the longitudinal axis of the catheter, the torroidal magnet having non-magnetic material between the opposite poles.

16. The system of claim 1, wherein the one or more magnetic sensors comprises a plurality of magnetic sensors.

17. The system of claim 1, wherein the one or more magnetic sensors are configured for mounting external to a patient's body.

18. The system of claim 1, further comprising one or more reference catheters, wherein the one or more magnetic sensors are mounted to the one or more reference catheters.

19. The system of claim 1, wherein the one or more magnetic sensors are selected from the group consisting of coil, Hall-effect and Giant Magnetoresistive (GMR) sensors.

20. The system of claim 1, wherein the processing circuitry is configured for further determining the positional coordinates of the catheter within a three-dimensional coordinate system based on the sensed magnetic field.

21. The system of claim 1, wherein the processing circuitry is configured for further determining the pitch and yaw of the catheter within the three-dimensional coordinate system based on the sensed magnetic field.

22. The system of claim 1, wherein the processing circuitry comprise a table with magnetic field intensities as inputs and the roll of the catheter as an output.

23. A method of tracking a catheter, comprising:
introducing the catheter into the body of a patient;
emitting a magnetic field from a permanent magnet mounted to the catheter, the magnetic field being asymmetrical about a longitudinal axis of the catheter;
sensing the magnetic field; and
determining the roll of the catheter about the longitudinal axis based on the sensed magnetic field.

24. The method of claim 23, further comprising performing a medical procedure with the catheter.

25. The method of claim 24, wherein the medical procedure is ablating tissue.

26. The method of claim 24, wherein the medical procedure is mapping cardiac tissue.

27. The method of claim 24, wherein the medical procedure is imaging tissue.

28. The method of claim 24, wherein the medical procedure is delivering a therapeutic agent to tissue.

29. The method of claim 23, further comprising calibrating the one or more permanent magnets.

30. The method of claim 23, wherein the magnetic field is sensed external to the body of the patient.

31. The method of claim 23, wherein the magnetic field is sensed internal to the body of the patient.

32. The method of claim 23, further comprising determining the positional coordinates of the catheter within a three-dimensional coordinate system based on the sensed magnetic field.

33. The method of claim 32, further comprising determining the pitch and yaw of the catheter within the three-dimensional coordinate system based on the sensed magnetic field.

34. The method of claim 23, further comprising storing a table having magnetic field intensities as inputs and the roll of the catheter as an output, wherein the determined roll of the catheter is based further on the table.
